(19) United States
(12) Patent Application Publication
(10) Pub. No.: US 2023/0207053 A1
(43) Pub. Date: Jun. 29, 2023

(54) Adjusted Polygenic Risk Score Calculation Algorithm and Process

(71) Applicant: The Scripps Research Institute, La Jolla, CA (US)

(72) Inventors: Ali Torkamani, San Diego, CA (US); Nathan Wineinger, San Diego, CA (US)

(21) Appl. No.: 17/998,750

(22) PCT Filed: May 14, 2021
(86) PCT No.: PCT/US21/32524

§ 371 Date: Nov. 14, 2022

Related U.S. Application Data
(60) Provisional application No. 63/025,560, filed on May 15, 2020.

(51) Int. Cl. G16B 20/20; G16B 20/30; G16B 40/20; G16H 50/30

(52) U.S. Cl. CPC G16B 20/20; G16B 20/30; G16B 40/20; G16H 50/30

(57) ABSTRACT

The invention disclosed herein relates to methods for estimating an individual's genetic risk to a specific phenotypic trait.

### CLAIM OF PRIORITY UNDER 35 U.S.C. § 119

The present application for patent claims priority to Provisional Application No. 63/025,560 entitled “ADJUSTED POLYGENIC RISK SCORE CALCULATION ALGORITHM AND PROCESS” filed May 15, 2020, which is hereby expressly incorporated by reference herein.

### BACKGROUND

### Field

The invention disclosed herein relates to methods for estimating an individual's genetic risk to a specific phenotypic trait.

### Background

Genetic risk for common heritable human (and non-human) diseases, conditions, and traits can be estimated with a polygenic risk score (PRS)—also referred to as genetic risk scores, polygenic scores, and genome-wide (risk) score. Genetic risk scores are most commonly calculated as a weighted sum of the number of risk alleles carried by an individual, where the risk alleles and their weights are defined by the loci and their measured effects as detected by genome-wide association studies (GWAS) (1) (see, e.g., US Patent Application 20190017119, incorporated herein by reference in its entirety). In some instances, a lower threshold than genome-wide statistical significance may be used to improve or estimate total predictability, often at the expense of generalizability (2-4). In other instances, models may be recalibrated to account for biases in effect size that are typically inflated in the discovery cohort, to account for multiple linked variants within each disease associated locus, to re-estimate effect sizes for a sub-phenotype of interest, or to adjust for ethnic or demographic factors that may influence the generalizability of models (1,5). This invention relates to selecting variants for inclusion in PRSs and re-estimating variant effects and overall polygenic risk scores to account for genetic and/or environmental substructure, where environmental substructure is defined by similarities in geographical, demographic, clinical, behavioral, and/or any other measurable characteristics.

### SUMMARY

Some embodiments of the invention relate to a computer-implemented method of determining a likelihood that an individual has, or will develop, a specific phenotypic trait. The method can include: (a) obtaining genomic data from the individual; (b) comparing the genomic data from the individual to reference genomic data; (c) assigning a subpopulation of the individual; (d) determining a polygenic risk score (PRS) of the specific phenotype; (e) adjusting the PRS by the assigned subpopulation; and (f) calculating an adjusted PRS. The adjusted PRS can be indicative of the likelihood that the individual has, or will develop the specific phenotypic trait.

In some embodiments, the determining step can include selecting one or more variants for inclusion in the PRS wherein such inclusion reduces a need to adjust Xᵢ and wᵢ across populations.

In some embodiments, selection of one or more variants can include a comparison of linkage disequilibrium structure between the individual's assigned subpopulation and the reference genomic data.

In some embodiments, selection of one or more variants can include prioritization based upon putative causal relationship to a trait of interest.

In some embodiments, the putative causal relationship can be identified by at least one variant interpretation process.

In some embodiments, the at least one variant interpretation process can include at least one of prior knowledge, position relative to, or influence on functional elements, influence on gene expression, prediction of functional impact, and/or the like, and/or any variant annotation category listed in FIGS. 2-3.

In some embodiments, the assigning of the subpopulation of the individual can be based on step (b) wherein the subpopulation is a population with at least 50% genetic similarity to the individual.

In some embodiments, the subpopulation can be a population with at least 80% genetic similarity to the individual.

In some embodiments, the subpopulation can be a population with at least 95% genetic similarity to the individual.

In some embodiments, the assigning of the subpopulation of the individual can be based on one or more environmental similarity. Environmental similarities can include similarities in geographical, demographic, clinical, behavioral, and/or any other measurable characteristics.

In some embodiments, the subpopulation can be a population within the same continent of the individual.

In some embodiments, the subpopulation can be a population within the same country or region of the individual.

In some embodiments, the subpopulation can be a population within the same city of the individual.

In some embodiments, the subpopulation can be a population of similar age, gender, and/or clinical diagnosis of the individual.

In some embodiments, the subpopulation can be a population of similar lifestyle of the individual.

Some embodiments of the invention relate to a computing device for determining methods described herein. The computing device can include one or more processors.

Some embodiments of the invention relate to a smart phone application using any of the methods described herein.

### BRIEF DESCRIPTION OF THE DRAWINGS

FIG. 1 is a flow chart illustrating aspects of the method herein.

FIG. 2 is a diagram illustrating four levels of annotation that can be used in the variant interpretation process.

FIG. 3 is a diagram illustrating an example of the process flow of an annotation pipeline that can be included in the invention.

### DETAILED DESCRIPTION

The invention relates to determining genetic risk scores, such that:

$$P R S = {\sum }_{i} {w}_{i} {X}_{i} = {w}^{T} X ,$$

which relates to the sum of genotype Xᵢ at locus i, coded as (0, 1, or 2) for additive effects at the locus (and can also be coded as 0, 1 to model dominance/recessive effects), weighted by a corresponding factor wᵢ. This factor itself can be expressed as a linear combination of weighted variables, such that

$${w}_{i} = {\sum }_{any k} {θ}_{k} {Z}_{k} ,$$

or more generally in matrix notation PRS=(θ^{T}Z)^{T} X. In the simple case this factor can be the corresponding effect from a prior large-scale GWAS study: e.g., the log odds ratio for categorical/disease traits or the mean genotype difference for quantitative traits.

The weights then can correspond to a one-unit change in X (the genetic dosage—corresponding to the effect of going from genotype 0 to 1, or equivalently 1 to 2) is the inverse function of the beta coefficient in a generalized regression model ƒ(Y)=g(βx) where Y is some trait and ƒ and g are functions. Thus, weights in the PRS are such that w=ƒ_{Y}⁻¹(y)=\hat{β}.

By design then,

$$P R S = {\sum }_{i} {\hat{β}}_{i} {X}_{i}$$

in the simple case which is what would be the estimate of a multivariable logistic regression of a categorical trait if all loci were conditionally independent with each with respect to disease risk:

$${log}_{e} ( disease odds ) ∼ PRS = {\sum }_{i} {\hat{β}}_{i} ( {X}_{i} ) .$$

Using this formula, each \hat{β} is an estimate with some standard error that decreases with sample size. For PRS calculation, \hat{β} can be determined in one reference population and applied to other populations. Populations can refer to genetic ancestry, but can also include populations defined by clustering of individuals by any spatial, demographic, behavioral, health status, genetic factors, and/or any other characteristics.

The invention relates to two considerations when applying this model to populations beyond the reference population: 1) the distribution of Xᵢ may differ across populations (i.e., different allele frequencies); and 2) the weight wᵢ, estimated by \hat{β} may differ between populations. Both will distort the interpretation of the PRS.

The invention relates to adjusting the above PRS to control for differences in wᵢ and the distribution of Xᵢ across populations. The output PRS for an individual based on the PRS distribution in a reference population matched to that individual can be standardized (population standardization), and/or the individual summed components of the PRS wᵢXᵢ by adjusting wᵢ or Xᵢ can be corrected (factor correction).

As used herein, “matched” and “assigned” can be used interchangeably.

### Population Standardization

In some embodiments of the invention “population standardization” is applied.

To perform population standardization, a matched population is identified in a variety of ways to standardize the overall PRS.

To control for genetic substructure, the individual's genome, X, is compared to the genomes of a population X̅ to define a genetically similar subpopulation. Genetic similarity can be defined globally across the entire genome, by a subset of ancestry informative markers, or can be defined by sets of variants defining polygenic risk scores or other genetic characteristics. A matched subpopulation is defined by one or many of these genetic similarity metrics and a clustering/grouping technique. The calculated PRS of an individual can then be standardized to the distribution of PRSs in the matched subpopulation. The individual's calculated PRS is standardized to the distribution of PRSs in the matched subpopulation.

To control for environmental substructure, an individual's environment, E, is compared to the environment of a population Ē, to define an environmentally similar subpopulation. Environmental similarity can be defined by one or more geographical characteristics, demographic characteristics, risk factor characteristics, behavioral characteristics, metabolic characteristics, and/or any other measurable characteristics. A matched subpopulation is defined by one or many of these environmental similarity metrics and a clustering/grouping technique. Thus, an environmental substructure can be defined by having similarities in geographical, demographic, clinical, behavioral, and/or any other measurable characteristics. The individual's calculated PRS is standardized to the distribution of PRSs in the matched subpopulation. “Similar” and “similarity” can be defined, in some embodiments, by having at least plus or minus 50% of the quantitative measure. In other embodiments, where noted as such, similarity can be quantitatively limited to plus or minus 40%, 30%, 25%, 20%, 15%, 10%, or 5%.

### Factor Correction

In some embodiments of the invention, factor correction is applied.

To perform individual factor correction, a matched population is identified in a variety of ways to correct for population differences in wᵢ and the distribution of Xᵢ;

To control for overall genetic substructure, the individual's genome, X, is compared to the genomes of a population X̅ to define a genetically similar subpopulation. Genetic similarity can be defined globally across the entire genome, by a subset of ancestry informative markers, or can be defined regionally using the genetic information surrounding each locus entered into the PRS calculation. A matched subpopulation is defined by one or many of these genetic similarity metrics and/or a clustering/grouping technique. For factor correction, the individual components of the PRS calculation for the individual can then be corrected using this matched subpopulation;

To correct for differences in the distribution of Xᵢ across subpopulations Xᵢ for the average genotype in their matched subpopulation X̅ᵢ is corrected at each locus i and its estimated standard deviation ŝᵢ:

$${X}_{i}^{*} = \frac{( {X}_{i} - {\overline{X}}_{i} )}{{\hat{s}}_{i}}$$

An environmentally similar subpopulation can be defined by comparing an individual's environment, E, to the environment of a population Ē. Environmental similarity, as described previously, can be defined by one or more geographical characteristics, demographic characteristics, behavioral characteristics (e.g., culture, lifestyle, and other social factors), risk factor characteristics, metabolic characteristics, and/or any other measurable characteristics. A matched subpopulation is defined by one or many of these environmental similarity metrics and a clustering/grouping technique. As above, Xᵢ for the average genotype in their matched subpopulation X̅ᵢ is corrected at each locus i.

Both genetically-defined and environmentally-defined subpopulations can also be used to correct for differences in wᵢ across subpopulations. A genetically- or environmentally-matched subpopulation is defined as described above, and \hat{β} is re-estimated using only individuals from the matched subpopulation as described in the Introduction for each locus i.

In some embodiments, this approach takes into account genetically-matched (ancestral) subpopulations with a genetic match at or above 50%. In other embodiments, subpopulations have a genetic match of at least 80%. In still other embodiments, the genetic match is 95% or higher. Likewise, in some embodiments, the approach takes into account environmentally-matched subpopulations of individuals residing in a political, geographic, or climatic zone or boundary of less than a continent, or determined to share similar environments through similarities in behavioral, clinical, demographic, or other measurable characteristics. In other embodiments, subpopulations are defined as individuals living within boundaries of less than a country or region (e.g., northern Europe vs. southern Europe or west Asia vs. east Asia, etc.). In further embodiments, the subpopulation is defined as individuals living within an area no larger than a city, a county, a valley, a climate zone, or other shared characteristic capable of distinguishing individuals with a relatively high level of shared environmental factors that are distinguishable from the environmental factors, as a whole, experienced by individuals outside the subpopulation.

In some embodiments, when such data are available, matched subpopulations are further stratified according to other relevant environmental factors including but not limited to: (a) differentiation between urban, suburban, and rural location and lifestyle; (b) differentiation by socioeconomic class within a defined geographic location (which adjusts for meaningful environmental differences that can be associated with living conditions even among people who are in relatively close physical proximity); (c) differentiation based upon length of time an individual has resided within the defined boundaries, such that individuals having a longer residence time are weighted in the analysis and/or individuals having a shorter residence time are de-weighted; (d) age of the individuals within a geographic subpopulation; (e) gender; (f) body mass index; (g) lifestyle factors such as but not limited to (1) levels of activity; (2) diet; (3) sleep; (4) smoking status; (5) alcohol consumption; (h) measurement of clinical risk factors proximal to overt disease onset, such as but not limited to (1) blood pressure levels, (2) blood chemistries; (3) biomarkers indicative of ongoing disease processes; (i) ascertainment of environmental exposures, such as but not limited to (1) air pollution; (2) heavy metals and other environmental toxins; and (3) family history. Further factors are provided in Torkamani, Ali et al. “High-Definition Medicine.” Cell vol. 170, 5 (2017): 828-843. doi:10.1016/j.cell.2017.08.007, which is fully incorporated by reference in its entirety herein.

In some embodiments, when such data are available, the PRS is further corrected according to other relevant factors including but not limited to all the factors listed above.

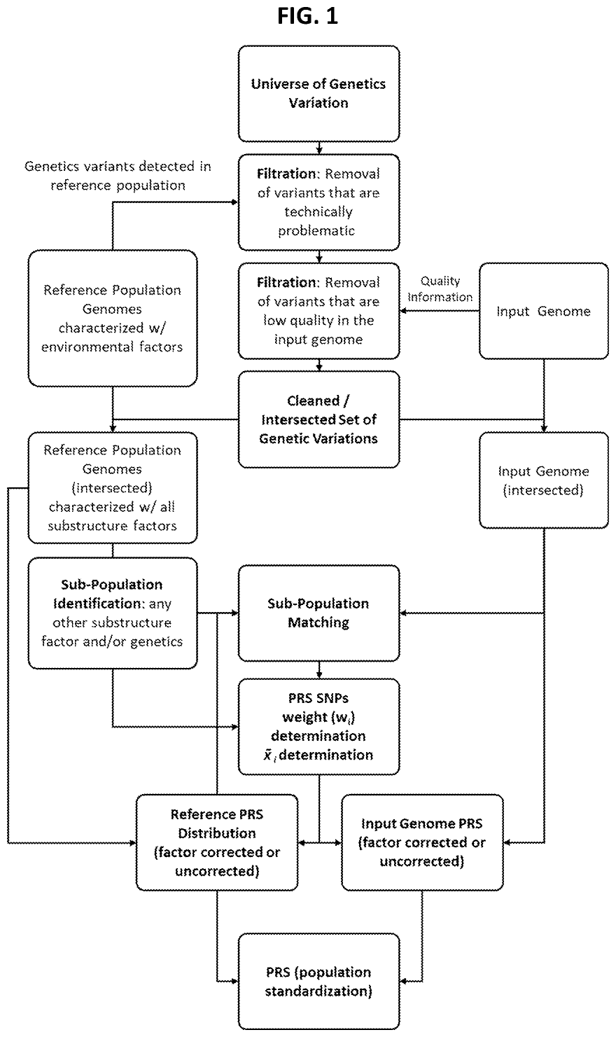

FIG. 1 helps to illustrate the methods described herein. As depicted in FIG. 1, the method can include obtaining an individual's genomic data (“Input Genome” in FIG. 1). These data can be from a service, such as 23 and Me, or the like. According to the invention, the data can be any source of genomic information from a heterogenous sampling of the human population.

In some embodiments, the method can include cleaning the individual's input genomic data by, for example, removing low quality variants as a result of sequencing inaccuracies, genotyping inaccuracies, genetic imputation inaccuracies, or other indicators of low quality genetic data acquisition, and/or the like (“Filtration: removal of variants that are low quality in the input genome” in FIG. 1”). Further descriptions can be found in Chen, S F., Dias, R., Evans, D. et al. Genotype imputation and variability in polygenic risk score estimation. Genome Med 12, 100 (2020). https://doi.org/10.1186/s13073-020-00801, which is hereby incorporated by reference in its entirety.

In some embodiments, the method includes cleaning all genetic variants (“Universe of Genetics Variation” in FIG. 1) under consideration by, for example, removing unnecessary information (e.g., chrX, chrY, mitochondrial DNA, etc.), removing genetic variants known to be reside in regions of the genome problematic for sequencing or genotyping assays, removing variants that are ambiguous in terms of strand orientation, and/or the like (“Filtration: removal of variants that are technically problematic” in FIG. 1”).

In some embodiments, the method includes matching the clean data index with reference genomic data (“Reference Population Genomes characterized w/environmental factors” in FIG. 1). The sequence can be from any large biobank with matched genomic and phenotypic data, such as UK Biobank or the like. Variant selection and wᵢ and Xᵢ for factor correction using the matched sub-population as described above (“PRS SNPs weight (wᵢ) determination Xᵢ determination”, in FIG. 1). wᵢ and Xᵢ factor correction can be performed using a different matched sub-population for each genetic variant included in the PRS.

In some embodiments, this approach selects variants for inclusion in the PRS that minimize the adjustments needed to Xᵢ and wᵢ across populations. To select variants that are generalizable for risk scoring across populations, variants are prioritized for inclusion in the PRS if their correlation structure with nearby genetic variants (known as “linkage disequilibrium” structure) is similar across the reference population and the individual's subpopulation.

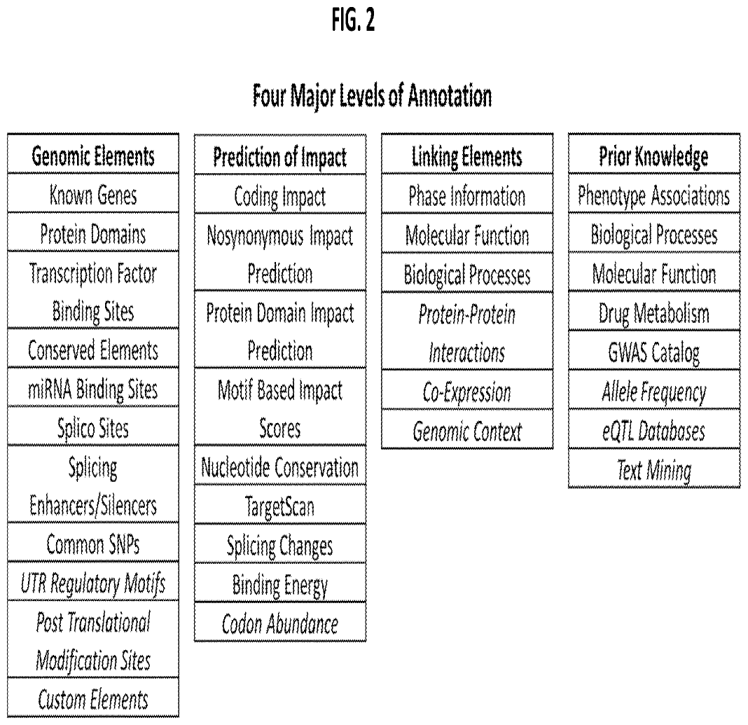

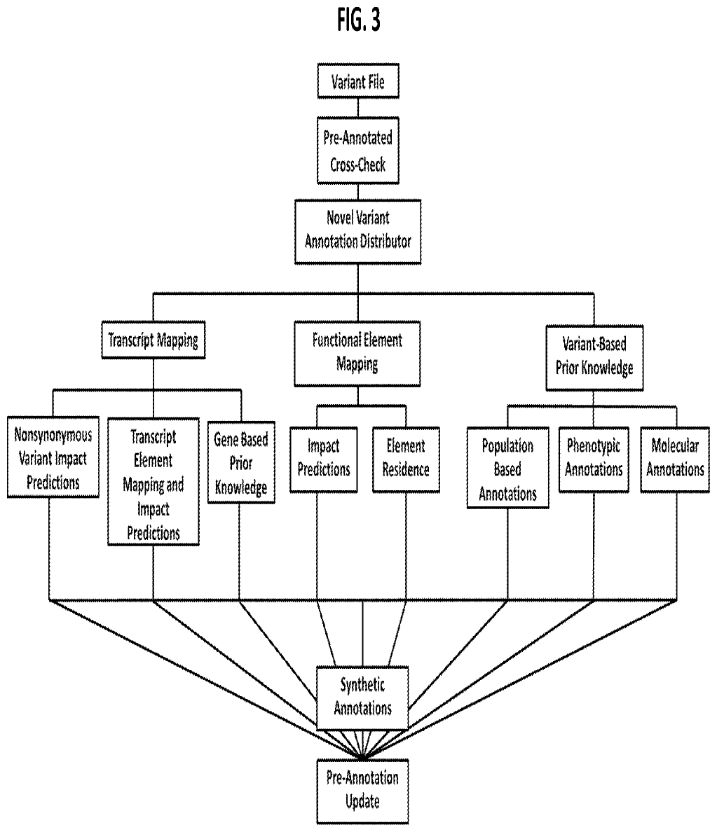

In some embodiments, this approach selects variants that are more likely to be causally related to the phenotypic trait of interest, reducing the need to adjust Xᵢ and wᵢ across populations. To select variants that are likely causal, variants are prioritized for inclusion in the PRS if they are deemed to be likely functional by variant interpretation processes. Variant annotation categories used as variant interpretation processes can include those provided in FIGS. 2 and 3.

Variant interpretation processes and other systems and method for prioritizing variants used in the invention can be found in U.S. application Ser. No. 16/351,394, entitled “Systems and methods for genomic annotation and distributed variant interpretation” and filed Mar. 12, 2019, the entire content of the foregoing is fully incorporated by reference herein.

For example, the variant interpretation process can include a computer-based genomic annotation system. The process can include a database configured to store genomic data, non-transitory memory configured to store instructions, and at least one processor coupled with the memory, the processor configured to implement the instructions in order to implement an annotation pipeline and at least one module for filtering or analysis of genomic data.

In some embodiments, the method can include calculating a factor-corrected or uncorrected reference genome PRS distribution (“Reference PRS Distribution (factor corrected or uncorrected)”, in FIG. 1).

In some embodiments, the method can include calculating a factor-corrected or uncorrected input genome PRS (“Input Genome PRS (factor corrected or uncorrected)”, in FIG. 1).

In some embodiments, the method can include calculating a population standardized input genome PRS by determining the percentile rank of the Input Genome PRS to the Reference PRS Distribution.

### EXAMPLES

### Example 1

In this example, the method accounts for statistical biases in the PRS with respect to the individual's underlying genetic background or ancestry by comparing the individual's PRS to those of a simulated sample customized to their genetic background. This information is returned to the user in the form of a percentile relative this sample; that is:

Pr(user's PRS>PRS_{Custom}),

where PRS_{Custom} is a list of sample PRSs. These sample PRSs can be constructed, rapidly, for any user from sets of (assumed) homogeneous populations with pre-calculated PRSs, PRS. In this example, 1000 Genomes reference samples are used as these populations. Thus:

PRS={PRS_{AFR},PRS_{AMR},PRS_{EAS},PRS_{EUR},PRS_{SAS}},

representative of the five continental super populations in 1000 Genomes. PRS_{Custom} is constructed by sampling a large number of times (e.g., 1 million) from the super populations within PRS, and weighting the k-th sample pre-calculated PRS,

PRSₖ={PRS_{AFR,k},PRS_{AMR,k},PRS_{EAS,k},PRS_{EUR,k},PRS_{SAS,k}},

by an appropriate weight v. That is

PRS_{custom}={v·PRSₖ|k=1,2, . . . }

Lastly, the weighting factor v represents the user's estimated genetic ancestry proportions in relation to the reference populations (e.g., 1000 Genomes). For example, if an individual is estimated to be 50% genetically African and 50% genetically European, PRS_{Custom} will consist of equal contribution from African and European ancestries. In this example,

PRS_{Custom}={0.5(PRS_{AFR,k})+0.5(PRS_{EUR,k})|k=1,2, . . . }.

As a result, biases due to population-level differences in PRSs with respect to genetic ancestry are eliminated. This approach heavily relies on markers contributing independent, additive effects across a genome. Additionally, the approach to a lesser extent assumes genetic markers contribute to traits evenly across populations. In other analyses, assumptions of even genetic contributions are removed and replaced with weighting of different markets, where such data are available with a meaningful sample size.

### Example 2

Genotype and phenotype data were obtained on the ARIC cohort through data access from dbGaP (phs000280). Imputation was performed on genetic data using minimac and reference haplotypes from the Haplotype Reference Consortium. CAD events were defined previously by ARIC study investigators. Sex, race identification, and age were collected from the first study visit data. The ARIC sample consisted of 13,214 individuals: 9,825 (74.3%) self-identified as white and 3,389 black; 7,238 (54.8%) women and 5,976 men; and with an average age at first study visit of 54.1 years (SD=5.7). Over the course of this study, 2,382 of these people (18.0%) had a CAD event.

A PRS was determined across the entire cohort, as well as separately based on shared characteristics, in this case for individuals of self-reported white or black ancestry. PRS weights were defined using logistic regression as described previously, using genetic variants known to be associated with CAD from prior GWAS studies. The percentile PRS, as defined in Example 1, was calculated for each study individual. These values were binned into low (0-20 percentile), average (20-80), and high (80-100) risk categories. PRSs displayed divergent predictive power depending upon the population they are derived from and applied to.

### Example 3

Genotype and phenotype data were obtained from the UK Biobank. Imputation was performed on genetic data using minimac and reference haplotypes from the Haplotype Reference Consortium. Numerous lifestyle factors including job type, shiftwork, alcohol consumption, cigarette use, speeding tickets, and many other lifestyle factors were used to define environmental similarity through determination of the Euclidean distance between all UK Biobank individuals using comprehensive lifestyle data. Personalized PRSs are defined for each individual in the UK Biobank by identifying the 100,000 most environmentally similar individuals and performing genome-wide association study regression analysis to derive a PRS as previously described.

### Example 4

Genotype and phenotype data were obtained and environmental similarity determined as described in Example 3. For each individual their local genetic ancestry was determined for genomic loci included in a previously defined CAD PRS, derived, as described in either Example 2 or 3. The factors included in this PRS are then corrected by re-defining weights based on reference individuals sharing both environmental similarity as well as local genetic similarity for each variant included in the PRS.

### Example 5

Functional variants were defined by variant annotation process including the following:

### Variant Functional Element Mapping

All variants were mapped to the UCSC Genome Browser human reference genome, version hg18. Subsequently, variant positions were taken and their proximity to known genes and functional genomic elements was determined using the available databases available from the UCSC Genome Browser. Transcripts of the nearest gene(s) were associated with a variant, and functional impact predictions were made independently for each transcript. If the variant fell within a known gene, its position within gene elements (e.g. exons, introns, untranslated regions, etc.) was recorded for functional impact predictions depending on the impacted gene element. Variants falling within an exon were analyzed for their impact on the amino acid sequence (e.g. synonymous, nonsynonymous, nonsense, frameshift, in-frame, intercodon etc.).

### Variant Functional Effect Predictions and Annotations

Once the genomic and functional element locations of each variant site were obtained, a suite of bioinformatics techniques and programs to ‘score’ the derived alleles (i.e., derived variant nucleotides) were leveraged for their likely functional effect on the genomic element they resided in. Derived variants were assessed for potential functional effects for the following categories: nonsense SNVs, frameshift structural variants, splicing change variants, probably damaging non-synonymous coding (nsc) SNVs, possibly damaging nscSNVs, protein motif damaging variants, transcription factor binding site (TFBS) disrupting variants, miRNA-BS disrupting variants, exonic splicing enhancer (ESE)-BS disrupting variants, and exonic splicing silencer (ESS)-BS disrupting variants.

The functional prediction algorithms used exploit a wide variety of methodologies and resources to predict variant functional effects, including conservation of nucleotides, known biophysical properties of DNA sequence, DNA-sequence determined protein and molecular structure, and DNA sequence motif or context pattern matching.

### Genomic Elements and Conservation

All variants were associated with conservation information in two ways. First, variants were associated with conserved elements from the phastCons conserved elements (28 way, 44 way, 28 wayPlacental, 44 wayPlacental, and 44 wayPrimates). These conserved elements represent potential functional elements preserved across species. Conservation was also assessed at the specific nucleotide positions impacted by the variant using the phyloP method. The same conservation levels as phastCons were used in order to gain higher resolution into the potential functional importance of the specific nucleotide impacted by the variant.

### Transcription Factor Binding Sites and Predictions

All variants, regardless of their genomic position, were associated with predicted transcription factor binding sites (TFBS) and scored for their potential impact on transcription factor binding. Predicted TFBS was pre-computed by utilizing the human transcription factors listed in the JASPAR and TRANSFAC transcription-factor binding profile to scan the human genome using the MOODS algorithm. The probability that a site corresponds to a TFBS was calculated by MOODS based on the background distribution of nucleotides in the human genome. TFBS at a relaxed threshold within (p-value<0.0002) was labeled in conserved, hypersensitive, or promoter regions, and at a more stringent threshold (p-value<0.00001) for other locations in order to capture sites that are more likely to correspond to true functional TFBS. Conserved sites correspond to the phastCons conserved elements, hypersensitive sites correspond to Encode DNASE hypersensitive sites annotated in UCSC genome browser, while promoters correspond to regions annotated by TRANSPro, and 2 kb upstream of known gene transcription start sites, identified by SwitchGear Genomics ENCODE tracks. The potential impact of variants on TFBS were scored by calculating the difference between the mutant and wild-type sequence scores using a position weighted matrix method and shown to identify regulatory variants in.

### Splicing Predictions

Variants falling near exon-intron boundaries were evaluated for their impact on splicing by the maximum entropy method of maxENTscan. Maximum entropy scores were calculated for the wild-type and mutant sequence independently, and compared to predict the variants impact on splicing. Changes from a positive wild-type score to a negative mutant score suggested a splice site disruption. Variants falling within exons were also analyzed for their impact on exonic splicing enhancers and/or silencers (ESE/ESS). The numbers of ESE and ESS sequences created or destroyed were determined based on the hexanucleotides reported as potential exonic splicing regulatory elements and shown to be the most informative for identification of splice-affecting variants.

### MicroRNA Binding Sites

Variants falling within 3′UTRs were analyzed for their impact on microRNA binding in two different manners. First, 3′UTRs were associated with pre-computed microRNA binding sites using the targetScan algorithm and database. Variant 3′UTR sequences were rescanned by targetScan in order to determine if microRNA binding sites were lost due to the impact of the variation. Second, the binding strength of the microRNA with its wild-type and variant binding site was calculated by the RNAcofold algorithm to return a AAG score for the change in microRNA binding strength induced by introduction of the variant.

### Protein Coding Variants

While interpretation of frameshift and nonsense mutations is fairly straightforward, the functional impact of nonsynonymous changes and in-frame indels or multi-nucleotide substitutions is highly variable. The PolyPhen-2 algorithm, which performs favorably in comparison to other available algorithms, was utilized for prioritization of nonsynonymous single nucleotide substitutions. A major drawback to predictors such as PolyPhen-2 is the inability to address more complex amino acid substitutions. To address this issue, the Log R.E-value score of variants, which is the log ratio of the E-value of the HMMER match of PFAM protein motifs between the variant and wild-type amino acid sequences, were also generated. This score has been shown to be capable of accurately identifying known deleterious mutations. More importantly, this score measures the fit of a full protein sequence to a PFAM motif; therefore multinucleotide substitutions are capable of being scored by this approach.

The universe of variants determined to be functional using the various variant annotation strategies described above were selected and a PRS determined using the process described in Examples 2, 3, or 4.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

1. Chatterjee N, Shi J, Garcia-Closas M. Developing and evaluating polygenic risk prediction models for stratified disease prevention. Nature Reviews Genetics. 2016.
2. Chatterjee N, Wheeler B, Sampson J, Hartge P, Chanock S J, Park J H. Projecting the performance of risk prediction based on polygenic analyses of genome-wide association studies. Nat Genet. 2013;
3. Zhu Z, Bakshi A, Vinkhuyzen A A, Hemani G, Lee S H, Nolte I M, et al. Dominance genetic variation contributes little to the missing heritability for human complex traits. Am J Hum Genet [Internet]. 2015; 96(3):377-85. Available from: https://www.ncbi.nlm.nih.gov/pubmed/25683123
4. Dudbridge F. Power and Predictive Accuracy of Polygenic Risk Scores. PLoS Genet. 2013;
5. Vilhjalmsson B J, Yang J, Finucane H K, Gusev A, Lindstrom S, Ripke S, et al. Modeling Linkage Disequilibrium Increases Accuracy of Polygenic Risk Scores. Am J Hum Genet [Internet]. 2015; 97(4):576-92. Available from: https://www.ncbi.nlm.nih.gov/pubmed/26430803
6. Torkamani A, Wineinger N E, Topol E J. The personal and clinical utility of polygenic risk scores. Nature Reviews Genetics. 2018.

What is claimed is:

1. A computer-implemented method of determining a likelihood that an individual has, or will develop, a specific phenotypic trait, the method comprising:
a. obtaining genomic data from the individual;
b. comparing the genomic data from the individual to reference genomic data;
c. assigning a subpopulation of the individual;
d. determining a polygenic risk score (PRS) of the specific phenotype;
e. adjusting the PRS by the assigned subpopulation;
f. calculating an adjusted PRS;
wherein the adjusted PRS is indicative of the likelihood that the individual has, or will develop the specific phenotypic trait.

2. The method of claim 1, wherein the determining step comprises selecting one or more variants for inclusion in the PRS wherein such inclusion reduces a need to adjust Xᵢ and wᵢ across populations.

3. The method of claim 2, wherein selection of one or more variants comprises a comparison of linkage disequilibrium structure between the individual's assigned subpopulation and the reference genomic data.

4. The method of claim 2, wherein, selection of one or more variants comprises prioritization based upon putative causal relationship to a trait of interest.

5. The method of claim 4, wherein the putative causal relationship is identified by at least one variant interpretation process.

6. The method of claim 5, wherein the at least one variant interpretation process comprises at least one of prior knowledge, position relative to or influence on functional elements, influence on gene expression, prediction of functional impact.

7. The method of claim 1, wherein the assigning of the subpopulation of the individual is based on step (b) wherein the subpopulation is a population with at least 50% genetic similarity to the individual.

8. The method of claim 7, wherein the subpopulation is a population with at least 80% genetic similarity to the individual.

9. The method of claim 8, wherein the subpopulation is a population with at least 95% genetic similarity to the individual.

10. The method of claim 1, wherein the assigning of the subpopulation of the individual is based on environmental similarities, wherein the environmental similarities include similarities in geographical, demographic, clinical or geographical or demographic or clinical or behavioral similarities.

11. The method of claim 1, wherein the subpopulation is a population within the same continent of the individual.

12. The method of claim 1, wherein the subpopulation is a population within the same country or region of the individual.

13. The method of claim 1, wherein the subpopulation is a population within the same city of the individual.

14. The method of claim 1, wherein the subpopulation is a population of similar age, gender, and clinical diagnosis of the individual.

15. The method of claim 1, wherein the subpopulation is a population of similar lifestyle of the individual.

16. A computing device for determining the method of claim 1 comprising one or more processors.

17. A smart phone application using the method of claim 1.
